(19) United States
(12) Patent Application Publication
(10) Pub. No.: US 2024/0122466 A1
(43) Pub. Date: Apr. 18, 2024

(54) AN UNMANNED WEARABLE APPARATUS FOR MEASURING THE REFRACTIVE ERROR OF AN EYE

(71) Applicant: Maheswari SRINIVASAN, Chennai (IN)

(72) Inventor: Maheswari SRINIVASAN, Chennai (IN)

(21) Appl. No.: 18/277,588

(22) PCT Filed: May 4, 2022
(86) PCT No.: PCT/IN2022/050418

§ 371 Date: Aug. 17, 2023

(30) Foreign Application Priority Data
Jun. 27, 2021 (IN) 202141028827

(51) Int. Cl. A61B 3/028; A61B 3/00; A61B 3/103

(52) U.S. Cl. CPC A61B 3/028; A61B 3/0008; A61B 3/0025; A61B 3/103

(57) ABSTRACT

An unmanned wearable apparatus for measuring the refractive error of an eye is disclosed. Said apparatus comprises: a visual acuity testing unit that is configured to measure the visual acuity of a person wearing said apparatus by forming a final image at 6 meters; an objective refraction testing unit that is configured to measure the objective refraction of said person; a subjective refraction testing unit that is configured to measure the subjective refraction of said person; and a control member (110) that is configured to monitor and control the operations of said apparatus. Said apparatus is configured to automatically switch from one test to another. The disclosed apparatus: is portable; is unmanned (can be used without a medical professional's help); and helps save space.

### FIELD OF THE INVENTION

The present disclosure is generally related to an apparatus for measuring the refractive error of an eye. Particularly, the present disclosure is related to a wearable apparatus for measuring the refractive error of an eye. More particularly, the present disclosure is related to an unmanned wearable apparatus for measuring the refractive error of an eye.

### BACKGROUND OF THE INVENTION

Recent advances in technology and telecommunication have propelled the steady growth of telemedicine. Telemedicine is a trending field, especially in view of pandemics, such as COVID-19.

As the world begins to accept digital communication as the new normal, there is a requirement for measuring the refractive error of an eye as well. In the recent past, there have been attempts oriented towards online consultation and telemedicine options in all specialties of medicine. However, existing solutions in optometry require a trained optometrist to perform vision testing.

In eye testing, three tests are mandatory for the accurate measurement of refractive error and prescription of glasses. These are: visual acuity measurement; objective refraction; and subjective refraction. Typically, these three tests are performed separately.

U.S. Pat. No. 7,384,146 B2 discloses kiosks for automatic refraction. This automatic device performs eye examination without the presence of an optometrist and ophthalmologist. However, this patent discloses only auto refraction and not subjective refraction.

Auto refraction is only the objective refraction. The accuracy of objective refraction is questionable. This is because the accommodation of the human crystalline lens may cause inaccurate estimation of the refractive error. For this reason, optometrists always perform subjective refraction for getting accurate results.

There is, therefore, a need in the art for an unmanned wearable apparatus for measuring the refractive error of an eye, which overcomes the aforementioned drawbacks and shortcomings.

### SUMMARY OF THE INVENTION

An unmanned wearable apparatus for measuring the refractive error of an eye is disclosed. Said apparatus comprises: a visual acuity testing unit; an objective refraction testing unit; a subjective refraction testing unit; and a control member.

Said visual acuity testing unit is configured to measure the visual acuity of a person wearing said apparatus by forming a final image at 6 meters. Said visual acuity testing unit comprises: a concave light refraction member; and a convex light refraction member.

A miniature erect virtual image is formed on the same side of an object at 3.16 cm from said concave light refraction member, upon the falling of light rays on said concave light refraction member. The image of said concave light refraction member is the object for said convex light refraction member.

Said objective refraction testing unit is configured to: measure the objective refraction of said person; and be clipped onto a display. Said objective refraction testing unit comprises: a prism; a light refraction member; a light source; and an image capturing unit.

Light rays from said light source fall on said light refraction member. Parallel light rays emanating from said light refraction member fall on said prism, with said light rays being turned 90 degrees from the direction of their original path.

Said image capturing unit captures the light reflex reflected from the retina of the person's eye, with said captured data being transmitted to a processing unit.

Said subjective refraction testing unit is configured to measure the subjective refraction of said person. Said subjective refraction testing unit comprises: said concave light refraction member; said convex light refraction member; an at least a focusing light refraction member; and a gear system.

Said at least one focusing light refraction member is disposed in front of the eye, with the gear system controlling the disposing of said at least one focusing light refraction member.

Said control member is communicatively associated with: said light source; said image capturing unit; said gear system; and said display. Said control member is configured to monitor and control the operations of said apparatus, with said apparatus being configured to automatically switch from one test to another.

In an embodiment of the present disclosure, said apparatus is configured as a virtual reality headset. The overall length of said virtual reality headset may be as low as 15 cm. Said apparatus is powered by a suitable power source (for example, a rechargeable battery).

The disclosed apparatus offers the following advantages: it is portable; it is unmanned (can be used without a medical professional's help); and helps save space.

### BRIEF DESCRIPTION OF THE DRAWINGS

FIG. 1 illustrates an embodiment of a conventional Snellen chart, in accordance with the present disclosure;

FIG. 2 illustrates an embodiment of a conventional ETDRS (Early Treatment Diabetic Retinopathy Study) chart, in accordance with the present disclosure;

FIG. 3 illustrates the testing of left eye visual acuity, in accordance with an embodiment of the present disclosure;

FIG. 4 illustrates the testing of right eye visual acuity, in accordance with an embodiment of the present disclosure;

FIG. 5 illustrates the construction of a visual acuity testing unit of an unmanned wearable apparatus for measuring the refractive error of an eye, in accordance with an embodiment of the present disclosure;

FIG. 6 illustrates the method of functioning of a visual acuity testing unit of an unmanned wearable apparatus for measuring the refractive error of an eye, in accordance with an embodiment of the present disclosure;

FIG. 7 illustrates an example of visual acuity testing through an unmanned wearable apparatus for measuring the refractive error of an eye, in accordance with the present disclosure;

FIG. 8 illustrates the construction of an objective refraction testing unit of an unmanned wearable apparatus for measuring the refractive error of an eye, in accordance with an embodiment of the present disclosure;

FIG. 9 illustrates the construction of an objective refraction testing unit of an unmanned wearable apparatus for measuring the refractive error of an eye, in accordance with an embodiment of the present disclosure;

FIG. 10 and FIG. 11 illustrate examples of objective refraction testing (plus powers) through an unmanned wearable apparatus for measuring the refractive error of an eye, in accordance with various embodiments of the present disclosure;

FIG. 12 illustrates an example of objective refraction testing (minus power) through an unmanned wearable apparatus for measuring the refractive error of an eye, in accordance with an embodiment of the present disclosure;

FIG. 13 illustrates the construction of a subjective refraction testing unit of an unmanned wearable apparatus for measuring the refractive error of an eye, in accordance with an embodiment of the present disclosure;

FIG. 14 illustrates the method of functioning of a subjective refraction testing unit of an unmanned wearable apparatus for measuring the refractive error of an eye, in accordance with an embodiment of the present disclosure;

FIG. 15 illustrates the configuration of an unmanned wearable apparatus for measuring the refractive error of an eye, in accordance with an embodiment of the present disclosure; and

FIG. 16 illustrates the processing of data captured by an image capturing unit, in accordance with an embodiment of the present disclosure.

### DETAILED DESCRIPTION OF THE INVENTION

Throughout this specification, the use of the words “comprise” and “include”, and variations such as “comprises”, “comprising”, “includes”, and “including” may imply the inclusion of an element or elements not specifically recited. Further, the disclosed embodiments may be embodied in various other forms as well.

Throughout this specification, the phrases “at least a”, “at least an”, and “at least one” are used interchangeably.

Throughout this specification, the use of the word “plurality” is to be construed as being inclusive of at least one.

Throughout this specification, the use of the word “apparatus” is to be construed as a set of technical components and/or units that are communicatively and/or operably associated with each other, and function together as part of a mechanism to achieve a desired technical result.

Throughout this specification, the use of the phrase “unmanned wearable apparatus” and its variations is to be construed as “a wearable apparatus that can be worn and used by a person without the help of another person (such as a medical practitioner)”.

Throughout this specification, the use of the phrase “application on a computing device” and its variations is to be construed as being inclusive of: application installable on a computing device; website hosted on a computing device; web application installed on a computing device; website accessible from a computing device; and web application accessible from a computing device.

Throughout this specification, the use of the phrase “computing device” and its variations are to be construed as being inclusive of: the Cloud; remote servers; desktop computers; laptop computers; mobile phones; smart phones; tablets; phablets; and smart watches.

Throughout this specification, the use of the words “communication”, “couple”, and their variations (such as communicatively) is to be construed as being inclusive of: one-way communication (or coupling); and two-way communication (or coupling), as the case may be.

Throughout this specification, the words “the” and “said” are used interchangeably with the same meaning.

Also, it is to be noted that embodiments may be described as a method depicted as a flow chart, a flow diagram, a dataflow diagram, a structure diagram, or a block diagram. Although the operations in a method are described as a sequential process, many of the operations may be performed in parallel, concurrently, or simultaneously. In addition, the order of the operations may be re-arranged. A method may be terminated when its operations are completed, but may also have additional steps not included in the figure(s).

An unmanned wearable apparatus for measuring the refractive error of an eye (also referred to as “apparatus”) is disclosed. The apparatus broadly comprises: a visual acuity testing unit; an objective refraction testing unit; and a subjective refraction testing unit.

The visual acuity testing unit is configured to measure the visual acuity of a person wearing the apparatus or the visual acuity of a user of the apparatus (also referred to as “person”).

Visual acuity is a measure of the gap-finding ability of the person; it gives information on the entire ocular media, the function of the retina, optic nerve, and visual cortex. A visual acuity test gives the baseline data for any clinical decision in ophthalmology.

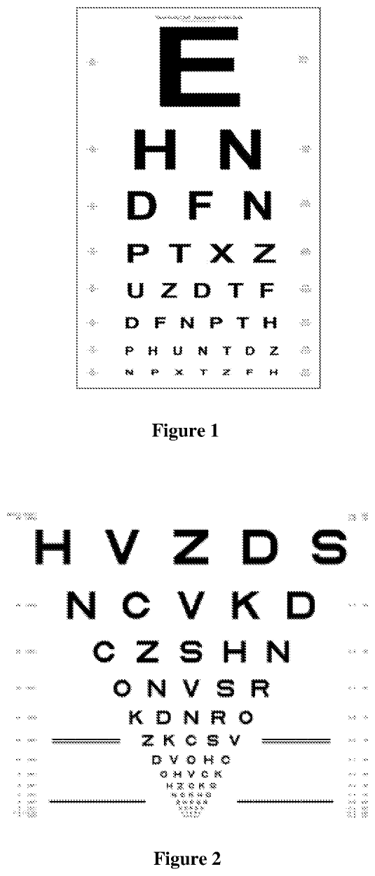

Visual acuity is conventionally tested through Snellen charts, an embodiment of which is illustrated in FIG. 1. Snellen defined the reference standard as the ability to recognize letters that are five minutes of arc high. However, population studies have shown that most healthy adults can outperform this standard.

Another conventional method of testing visual acuity is through Early Treatment Diabetic Retinopathy Study (ETDRS) charts, an embodiment of which is illustrated in FIG. 2. A line is considered to have been read if more than half of the letters (i.e. 3 of 5) are identified correctly.

The visual acuity testing unit tests the visual acuity of the person by presenting five optotypes for every size of a visual acuity line, through an output member. As recommended by the International Council of Ophthalmology, the testing distance is configured to be 6 meters.

Light Adaptation, luminance, and contrast levels of the display are configured to be as per the recommendations or guidelines of the International Council of Ophthalmology (120 cd/m²).

The size of the optotypes and progression of steps in optotypes are also configured, as per the recommendations or guidelines of the International Council of Ophthalmology.

In an embodiment of the present disclosure, the output member is a display (114; FIG. 13) with split-screen functionality. The display (114) may be of any type known in the art.

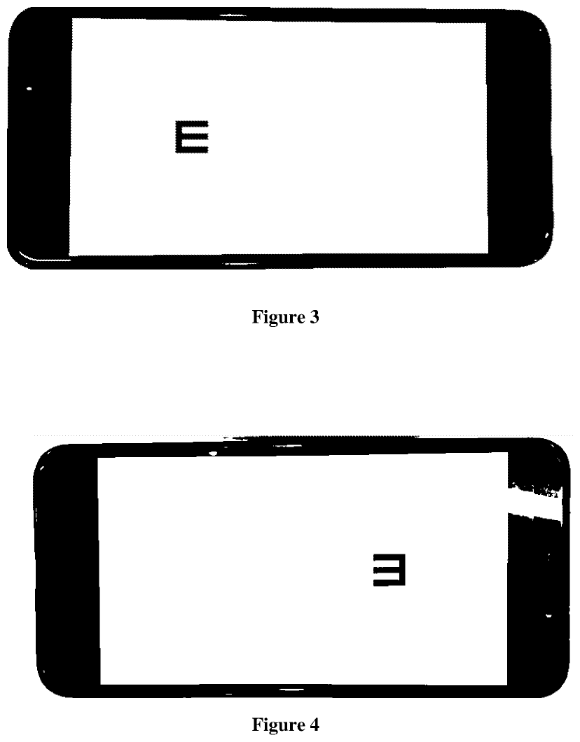

As illustrated in FIG. 3 and FIG. 4, the left eye of the person sees only the left display, while the right eye sees only the right display. At any point of the time, only one side of the display (114) displays the image. The display (114) is set according to the eye that is to be checked.

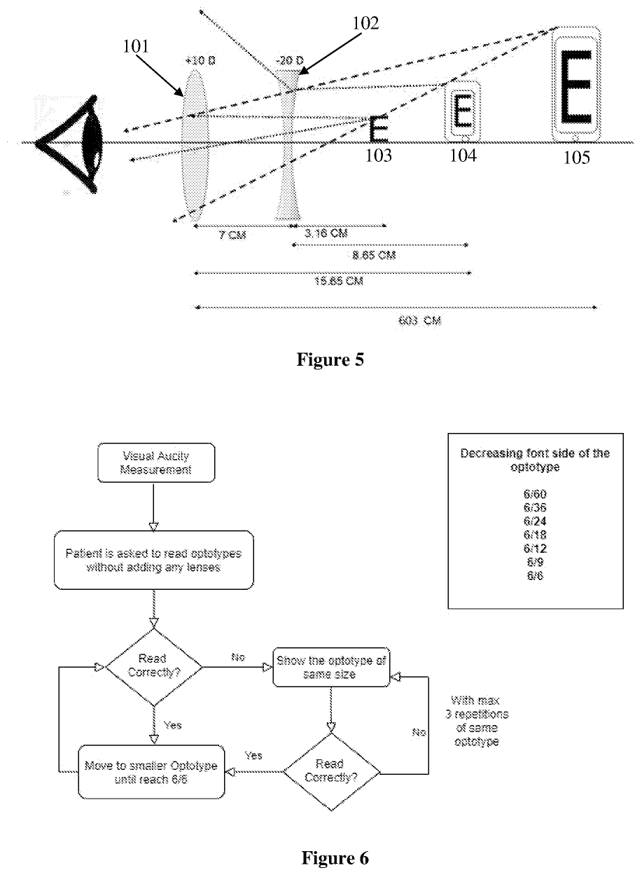

FIG. 5 illustrates the construction of the visual acuity testing unit. In another embodiment of the present disclosure, the visual acuity testing unit comprises: a concave light refraction member (102); a convex light refraction member (101); and a control member (110; FIG. 9).

The light rays travel from right to left, that is, the light rays emanate from the display (114) and travel towards the eye. The object on the display (104) is configured to be disposed 8.65 cm from the concave light refraction member (102), which is a −20 D lens.

The light rays fall on the concave light refraction member (102). This forms a miniature erect virtual image (103) on the same side of the object (104) at 3.16 cm from the concave light refraction member (102).

The image of the concave light refraction member (102) becomes the object for the convex light refraction member (101). Hence, the object distance for the convex light refraction member (101) is 10.16 cm. The convex light refraction member (101) is a +10 D lens, which results in the final image (105) being formed at 603 cm.

Therefore, the total length of the optical system is 15.65 cm.

This virtual distance of 6 meters behaves the same way as the physical distance of 6 meters in terms of its ability of form the image in the retina.

A person skilled in the art will appreciate the fact that the number of lenses and their configurations may vary.

The control member (110) is communicatively associated with the display (114), and is configured to monitor and control the operations of the apparatus.

In yet another embodiment of the present disclosure, the concave light refraction member (102) and the convex light refraction member (101) are made of allyl diglycol carbonate.

FIG. 6 illustrates the method of functioning of the visual acuity testing unit. In yet another embodiment of the present disclosure, the method of functioning of the visual acuity testing unit broadly comprises the following:

The person is asked to read a 6/60 optotype. If this is determined as read correctly by the control member (110), the visual acuity testing unit moves onto the next smaller optotype (6/36). If this is also read correctly, the visual acuity testing unit moves onto the next smaller optotype (6/24). This process is repeated until the smallest optotype (6/6).

At each optotype, the person is provided with a finite number of chances for reading the optotype (for example, three chances). The read optotype may be recognized through a voice recognition unit that comprises an audio capturing unit or an array of audio capturing units. The audio capturing unit may be of any type known in the art.

Since voice recognition units are well-known in the art, the features of the same are not disclosed herein.

Alternatively, or in addition to the above, the optotype may be typed through an input unit, which may be of any type known in the art.

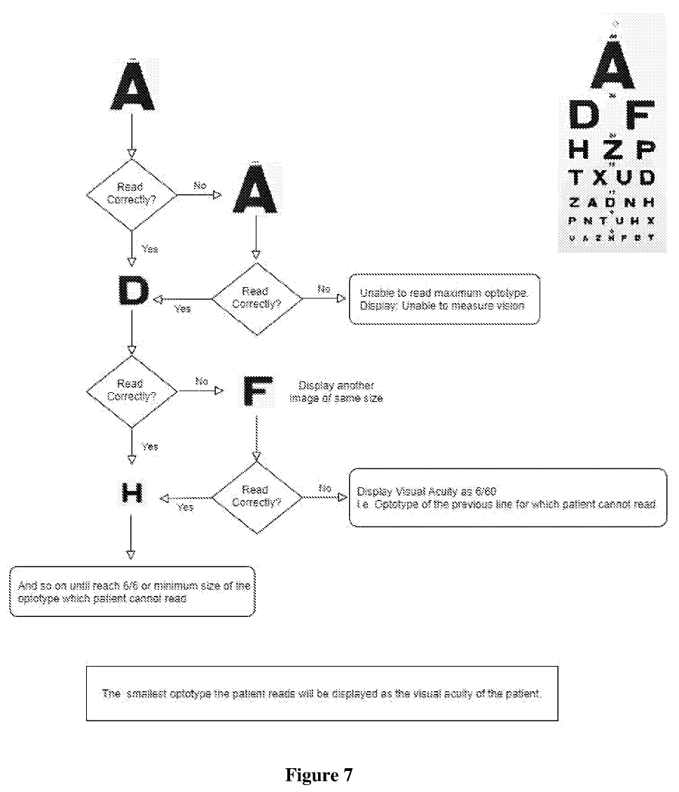

FIG. 7 illustrates an example of visual acuity testing through the apparatus. At the first step, the person is asked to read the letter “A” (6/60 optotype). If this is determined as read correctly by the control member (110), the person is asked to read the letter “D” (6/36 optotype).

If this is also determined as read correctly by the control member (110), in the next step, the person is asked to read the letter “H” (6/24 optotype). This process is repeated until the smallest optotype (6/6).

If the person is unable to read the letter “A” (6/60 optotype) in the first step, the person is asked to try again. If he/she succeeds in the second attempt, in the next step, the person is asked to read the letter “D” (6/36 optotype), and the process is repeated until the smallest optotype (6/6).

If, even after the exhaustion of the finite number of chances, the person is unable to read the letter “A” (6/60 optotype), the control member (110) instructs the display (114) to display “Unable to measure vision”.

In yet another embodiment of the present disclosure, the control member (110) is a single-board-computer (for example, a Raspberry Pi).

In yet another embodiment of the present disclosure, the control member (110) is a System on Chip (SoC).

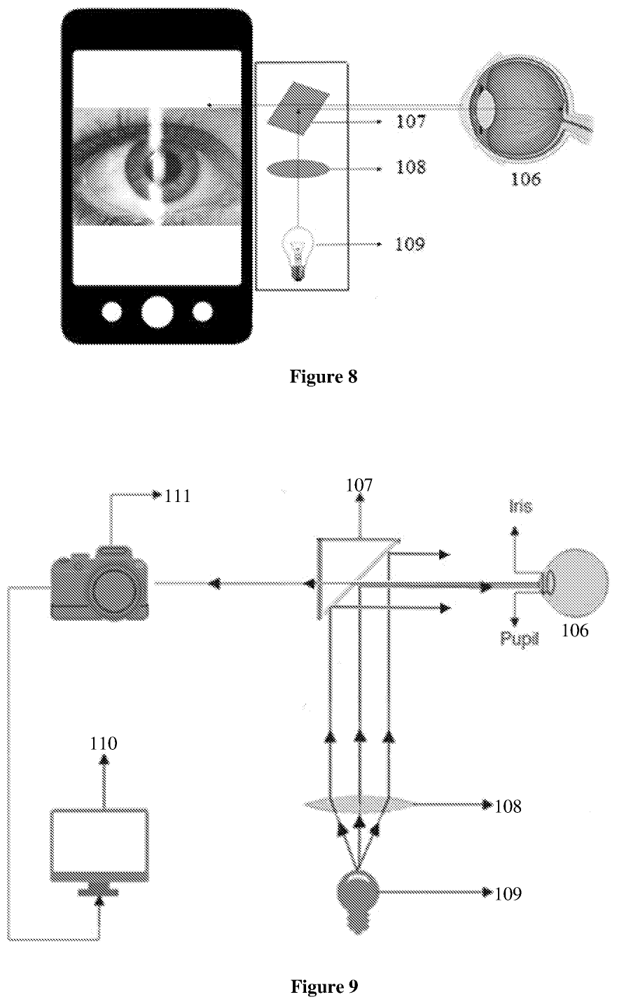

The objective refraction testing unit is configured to measure the objective refraction of the person. In yet another embodiment of the present disclosure, as illustrated in FIG. 8 and FIG. 9, the objective refraction testing unit comprises: a light source (109); a light refraction member (108); a 90-degree prism (107; also referred to as “prism”); an image capturing unit (111); and the control member (110). The objective refraction testing unit may be configured to be clipped onto the display (114).

The distance between the light source (109) and the light refraction member (108) is configured to be equivalent to the focal length of the light refraction member (108). For example, if the power of the light refraction member (108) is 20 D, the distance between the light refraction member (108) and light source (109) is 5 cm.

The light travels from the light source (109) and falls on the light refraction member (108). Since the light source (109) is kept at the focal length of the light refraction member (108), the light rays emanating from the light refraction member (108) are parallel. The parallel rays fall on the prism (107) and are turned 90 degrees from the direction of their original path. Thus, they fall on the eye (106).

In yet another embodiment of the present disclosure, the prism (107) is a totally internally reflecting prism made of glass.

In yet another embodiment of the present disclosure, the light refraction member (108) is a lens.

In yet another embodiment of the present disclosure, the light refraction member (108) is made of allyl diglycol carbonate.

In yet another embodiment of the present disclosure, the light source (109) is a bulb.

In yet another embodiment of the present disclosure, the image capturing unit (111) is a digital camera.

The control member (110) is communicatively associated with the image capturing unit (111), the display (114), and the light source (109).

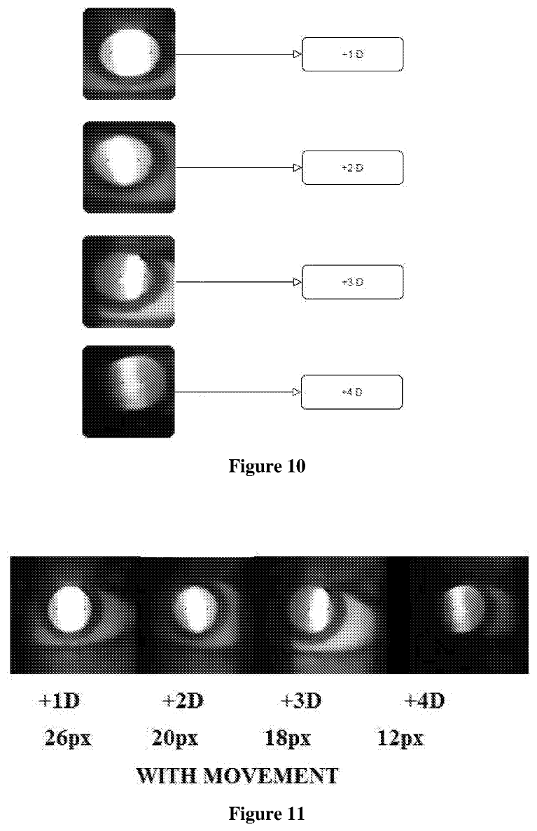

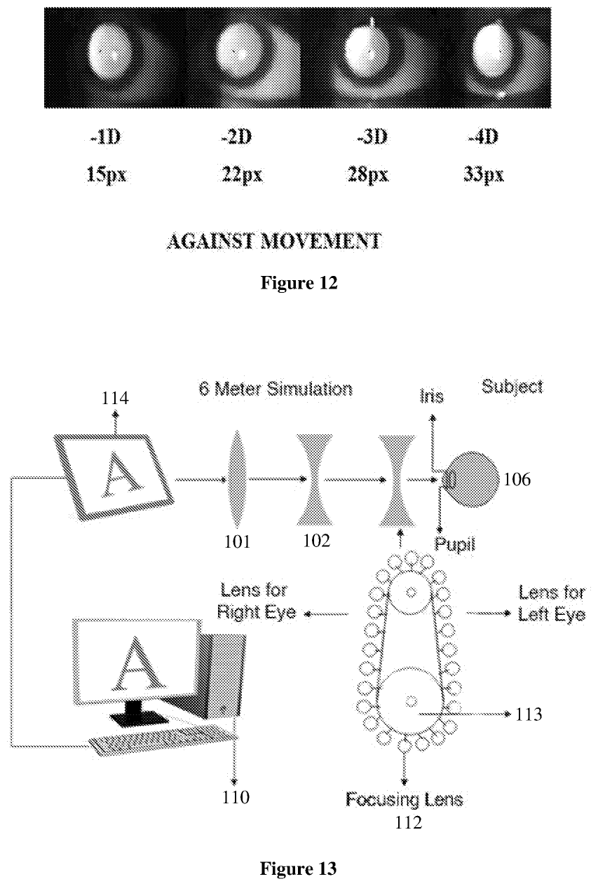

The light reflex reflected from the retina of the person's eye (106) is captured by the image capturing unit (111). The thickness and the intensity of the light reflex vary depending on the refractive error of the eye (106), as illustrated in FIG. 10, FIG. 11, and FIG. 12.

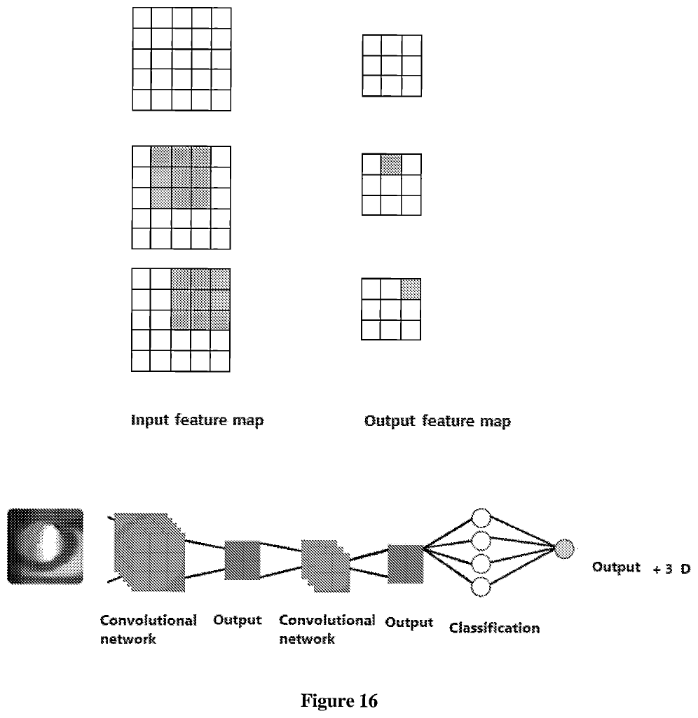

The captured data is/are transmitted to a processing unit, where it/they are processed and compared to pre-existing data for different refractive errors. As illustrated in FIG. 16, the segments of the captured data are studied and convolutional filter is created.

Based on the results of the comparison, the objective refraction is identified and transmitted to the objective refraction testing unit control member (110), which, in turn, instructs the display (114) to display the results.

The subjective refraction testing unit is configured to measure the subjective refraction of the person. In yet another embodiment of the present disclosure, as illustrated in FIG. 13, the subjective refraction testing unit comprises: the concave light refraction member (102); the convex light refraction member (101); the control member (110); an at least a focusing light refraction member (112); and a gear system (113) that is communicatively associated with the control member (110).

The at least one focusing light refraction member (112) is disposed in front of the eye (106), which corrects the refractive error of the eye (106). The power of the at least one focusing light refraction member (112) varies from one person to another person, and is determined by the subjective refraction testing unit.

Depending upon the refractive error, the at least one focusing light refraction member (112) is disposed in front of the eye (106). The disposing of the at least one focusing light refraction member (112) is controlled by the gear system (113), which may be of any type known in the art.

The light rays travel from the display (114) towards the eye (106). As explained earlier, this results in the projection of the image at 6 meters (FIG. 5). If there is no refractive error (no requirement of spectacle correction), the person will be able to read the optotypes on the display (114) till 6/6.

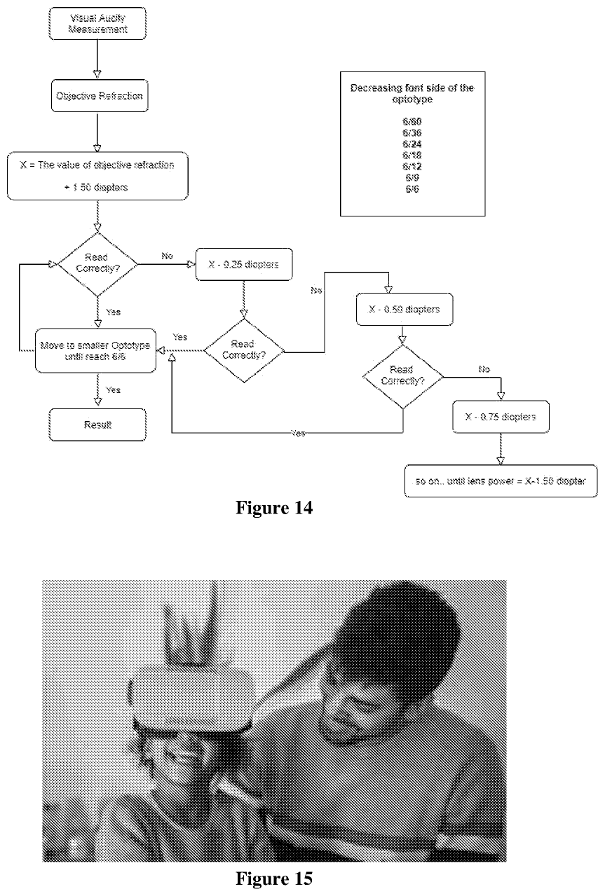

If the person has refractive error, he/she will not be able to read the optotypes till 6/6. In this case, the power of at least one focusing light refraction member (112) is equivalent to “X” (FIG. 14).

FIG. 14 illustrates the method of functioning of the subjective refraction testing unit. In yet another embodiment of the present disclosure, the method of functioning of the subjective refraction testing unit broadly comprises the following:

In the first step, the value of X is fixed at the value of objective refraction (arrived at through the objective refraction testing unit)+1.5 diopters. If this is determined as having been read correctly, the subjective refraction testing unit moves onto the next optotype. This process is continued until the smallest optotype.

On the other hand, if the person is unable to read at the first step, the value of X is reduced by 0.25 diopters. If the person is now able to read, the subjective refraction testing unit moves onto the next optotype. This process is continued until the smallest optotype.

If the person is unable to read even at X−0.25 diopters, the value of X is reduced by 0.5 diopters. If the person is now able to read, the subjective refraction testing unit moves onto the next optotype. This process is continued until the smallest optotype.

If the person is unable to read even at X−0.5 diopters, the value of X is reduced by 0.75 diopters. If the person is now able to read, the subjective refraction testing unit moves onto the next optotype. This process is continued until the smallest optotype.

If the person is unable to read even at X−0.75 diopters, the value of X is further reduced, and this process is continued until X−1.5 diopters.

The read optotype may be recognized through the voice recognition unit that comprises the audio capturing unit or the array of audio capturing units. The audio capturing unit may be of any type known in the art.

Alternatively, or in addition to the above, the optotype may be typed through the input unit, which may be of any type known in the art.

In yet another embodiment of the present disclosure, the apparatus is configured as a virtual reality headset, as illustrated in FIG. 15. Since a child is wearing the apparatus, an adult is shown as supervising the child in the illustration. The overall length of the virtual reality headset may be as low as 15 cm.

A person skilled in the art will appreciate the fact that common features of commercially available virtual reality headsets may be included in the virtual reality headset.

In yet another embodiment of the present disclosure, the apparatus is powered by a suitable power source (for example, a rechargeable battery). The apparatus also comprises a power button, through which the apparatus is switched on and switched off.

Alternately, or in addition to the rechargeable battery, the apparatus may also comprise a solar panel that powers the apparatus.

In yet another embodiment of the present disclosure, the apparatus comprises a master control unit that is communicatively associated with respective control members of the individual units.

The master control unit is configured to control and monitor the operations of the apparatus, while the control members of the individual units are configured to control and monitor the operations of the respective individual units.

In the above scenario, the control members of the individual units transmit signals to the master control unit, which, in turn, is communicatively associated with the display (114).

In yet another embodiment of the present disclosure, the master control unit and the control members of the individual units are single-board-computers (for example, a Raspberry Pi).

In yet another embodiment of the present disclosure, the master control unit and the control members of the individual units are System on Chip (SoC).

In yet another embodiment of the present disclosure, the apparatus comprises a communication member, through which the apparatus communicates with an application on a computing device. The apparatus may be configured and controlled remotely through the application on a computing device. A display of the computing device functions as an interface, through which the person or an operator interacts with the apparatus.

In addition to being configured to automatically switch from one test to another, the disclosed apparatus also offers the following advantages:
1. It is portable: It can be carried to camps, village, and can be used for home testing as well.
2. It is unmanned: It can be used without a medical professional's help. There is a huge burden of blindness in India and other developing countries. In this scenario, we have very limited manpower in the medical field.

The number of optometrists and ophthalmologists are significantly less when compared to the total population of India. With this apparatus, the only thing that optometrists and ophthalmologists have to do is the final decision-making based on the results obtained.
3. Space: In crowded cities like Chennai, Mumbai, etc., land is very expensive. A room with a 6 meter distance or a mirrored room with a 3 meters distance is very expensive. In this apparatus, the effect of a 6 meters distance is obtained by a portable apparatus.

Implementation of the apparatus and/or method of the disclosure can involve performing or completing selected tasks manually, automatically, or a combination thereof. Further, according to actual instrumentation of the apparatus and/or method of the disclosure, several selected tasks could be implemented by hardware, by software, by firmware, or by a combination thereof using an operating system.

For example, as software, selected tasks according to the disclosure could be implemented as a plurality of software instructions being executed by a computer using any suitable operating system.

In yet another embodiment of the disclosure, one or more tasks according to embodiments of the apparatus and/or method as described herein are performed by a data processor, such as a computing platform for executing a plurality of instructions.

Further, the data processor includes a processor, and/or non-transitory computer-readable medium for storing instructions and/or data, and/or a non-volatile storage for storing instructions and/or data. A network connection, a display, and/or a user input device such as a keyboard or mouse are also provided.

It will be apparent to a person skilled in the art that the above description is for illustrative purposes only and should not be considered as limiting. Various modifications, additions, alterations and improvements without deviating from the spirit and the scope of the disclosure may be made by a person skilled in the art. Such modifications, additions, alterations and improvements should be construed as being within the scope of this disclosure.

### LIST OF REFERENCE NUMERALS

101—Convex Light Refraction Member
102—Concave Light Refraction Member
103—Miniature Erect Virtual Image
104—Object on a Display or Object
105—Final Image
106—Eye
107—Prism
108—Light Refraction Member
109—Light Source
110—Control Member
111—Image Capturing Unit
112—At Least One Focusing Light Refraction Member
113—Gear System
114—Display

I claim:

1. An unmanned wearable apparatus for measuring the refractive error of an eye, comprising:
a visual acuity testing unit that is configured to measure the visual acuity of a person wearing said apparatus by forming a final image (105) at 6 meters, said visual acuity testing unit comprising:
a concave light refraction member (102), with a miniature erect virtual image (103) being formed on the same side of an object (104) at 3.16 cm from said concave light refraction member (102), upon the falling of light rays on said concave light refraction member (102); and
a convex light refraction member (101), with the image of said concave light refraction member (102) being the object for said convex light refraction member (101);
an objective refraction testing unit that is configured to: measure the objective refraction of said person; and be clipped onto a display (114), said objective refraction testing unit comprising:
a light source (109), from which light rays fall on a light refraction member (108);
a prism (107), onto which parallel light rays emanating from said light refraction member (108) fall, said light rays being turned 90 degrees from the direction of their original path; and
an image capturing unit (111) that captures the light reflex reflected from the retina of the person's eye (106), with said captured data being transmitted to a processing unit;
a subjective refraction testing unit that is configured to measure the subjective refraction of said person, said subjective refraction testing unit comprising: said concave light refraction member (102); said convex light refraction member (101); an at least a focusing light refraction member (112) that is disposed in front of the eye (106); and a gear system (113) that controls the disposing of said at least one focusing light refraction member (112); and
a control member (110) that is communicatively associated with: said light source (109); said image capturing unit (111); said gear system (113); and said display (114), said control member (110) being configured to monitor and control the operations of said apparatus, with said apparatus being configured to automatically switch from one test to another.

2. The unmanned wearable apparatus for measuring the refractive error of an eye as claimed in claim 1, wherein said concave light refraction member (102) is a −20 D lens.

3. The unmanned wearable apparatus for measuring the refractive error of an eye as claimed in claim 1, wherein said convex light refraction member (101) is a +10 D lens.

4. The unmanned wearable apparatus for measuring the refractive error of an eye as claimed in claim 1, wherein said concave light refraction member (102), said convex light refraction member (101), and said light refraction member (108) are made of allyl diglycol carbonate.

5. The unmanned wearable apparatus for measuring the refractive error of an eye as claimed in claim 1, wherein said control member (110) is: a single-board-computer; or a System on Chip.

6. The unmanned wearable apparatus for measuring the refractive error of an eye as claimed in claim 1, wherein said prism (107) is made of glass.

7. The unmanned wearable apparatus for measuring the refractive error of an eye as claimed in claim 1, wherein said apparatus is configured as a virtual reality headset of length 15 cm.
